(19) United States
(12) Patent Application Publication
(10) Pub. No.: US 2026/0027069 A1
(43) Pub. Date: Jan. 29, 2026

(54) Compositions Comprising Pramoxine and Diphenydramine for Treatment of Skin Conditions

(71) Applicant: VETNIQUE LABS, LLC, Naperville, IL (US)

(72) Inventor: Jamy A. Bascharon, Naperville, IL (US)

(21) Appl. No.: 18/994,516

(22) PCT Filed: Jul. 14, 2023
(86) PCT No.: PCT/US2023/027809

§ 371 Date: Jan. 14, 2025

Related U.S. Application Data
(60) Provisional application No. 62/389,029, filed on Feb. 16, 2016.

(51) Int. Cl. A61K 31/138; A61K 9/70; A61K 31/4166; A61K 31/5375; A61K 31/573; A61K 36/484

(52) U.S. Cl. CPC A61K 31/138; A61K 9/7007; A61K 31/4166; A61K 31/5375; A61K 31/573; A61K 36/484

(57) ABSTRACT

This disclosure relates to compositions for treating skin conditions and improving skin health. In particular, the disclosure relates to compositions formulated as an absorbent article to be topically applied to skin, the absorbent article comprising an analgesic agent, antihistamine agent and/or a corticosteroid.

### RELATED APPLICATION

This application claims priority to U.S. Provisional Application No. 62/389,029, filed Jul. 14, 2022, the contents of which are hereby incorporated by reference in their entirety.

### FIELD OF THE DISCLOSURE

This disclosure relates to compositions for promoting skin health benefits and for treating skin conditions. In particular, the disclosure relates to compositions formulated as an absorbent article comprising an analgesic agent, antihistamine agent and/or a corticosteroid. For example, the disclosure relates to compositions formulated as a wipe comprising pramoxine, diphenhydramine, and/or hydrocortisone.

### BACKGROUND OF THE DISCLOSURE

Promoting healthy, moisturized skin is important to maintaining the overall health of many mammals, including pets such as dogs and cats. Bacterial and fungal skin conditions can develop in such pets if their skin is not properly cleansed. Such skin conditions are generated by microorganism growth. Although microorganism growth can occur anywhere on the skin, regions of a pet's body that are particularly vulnerable and sensitive to microorganism growth are, for example, inside skin folds and around the ears, eyes, and nose.

The skin is a complex structure, and providing a beneficial effect on skin, including treatment of an adverse skin condition, by delivery of active substances or other ingredients or compositions for topical application, is associated with some limitations of effectiveness and subject comfort.

### SUMMARY OF THE DISCLOSURE

An absorbent article to be topically applied to a subject aids in treatment and prevention of various skin conditions, as well as promotes overall skin health. The specific combination of ingredients disclosed herein cleanses skin and treats skin conditions such as bacterial and fungal skin conditions. The specific combination of ingredients disclosed herein is adapted to adequately clean and treat various skin conditions, while not irritating sensitive skin areas such as around the ears, eyes, and nose, for example, of a subject.

One aspect of the disclosure provides a composition comprising an analgesic agent in the amount of about 0.1% to about 10% by weight of the total composition and an antihistamine agent in the amount of about 0.1% to about 10% by weight of the total composition.

Another aspect of the disclosure provides a composition comprising pramoxine in the amount of about 0.1% to about 10% by weight of the total composition and diphenhydramine in the amount of about 0.1% to about 10% by weight of the total composition.

Still another aspect of the disclosure provides a composition comprising pramoxine in the amount of about 1% by weight of the total composition, diphenhydramine in the amount of about 1% by weight of the total composition, and hydrocortisone is in the amount of about 0.5% by weight of the total composition.

In another aspect of the disclosure, a composition comprising pramoxine in the amount of about 0.5% by weight of the total composition, diphenhydramine in the amount of about 0.5% by weight of the total composition, and hydrocortisone is in the amount of about 0.1% by weight of the total composition.

In another aspect of the disclosure, a composition comprising pramoxine in the amount of about 1% by weight of the total composition, diphenhydramine in the amount of about 1% by weight of the total composition, and hydrocortisone is in the amount of about 0.5% by weight of the total composition, and one or more of propylene glycol, SD alcohol 40-B, glycerin, polysorbate 80, benzyl alcohol, PEG-75 lanolin, hydrolyzed oats, propanediol, glucose, glucuronic acid, and sodium hydroxide are provided.

Further provided herein is a method of treatment or prevention of a skin condition, the method comprising topically administering to a subject in need of such treatment an effective amount of one or more compositions disclosed herein.

### DETAILED DESCRIPTION OF THE DISCLOSURE

This disclosure relates to compositions for promoting skin health benefits and treating skin conditions. In particular, the disclosure relates to compositions formulated as an absorbent article, such as a wet wipe, comprising pramoxine, diphenhydramine hydrochloride, and/or hydrocortisone. Such a wipe kills and/or prevents the growth of bacteria on the skin, and treats fungi or scaly areas on the skin. Such a wipe additionally promotes skin health and does not irritate areas of the skin such as around the ears, eyes, and nose of a subject.

Ranges and amounts can be expressed as ‘about’ a particular value or range. About also includes the exact amount. For example, “about 5%” means “about 5%” and also “5%.” The term “about” can also refer to +10% of a given value or range of values. Therefore, about 5% also means 4.5%-5.5%, for example.

The term “treating” or “treatment” is used herein, for instance, in reference to methods of treating a skin disorder or condition, and generally includes the administration of a compound or composition which reduces the frequency of, or prevents the onset of, symptoms of a medical condition or enhances the condition or hydration of the intended skin treatment area in a subject relative to the subject not receiving the compound or composition. This can include reversing, reducing, or arresting the symptoms and underlying pathology of a condition. Such reversal, reduction, or stoppage of symptoms improves or stabilizes a subject's condition. Treating or treatment can include routine application for maintenance of cleansed and healthy skin.

The term “subject” is used herein to refer to a mammalian subject.

Pramoxine (CAS RN: 140-65-8) is an anesthetic used on the skin for pain, itching, and discomfort. In some embodiments, the disclosure provides a composition, wherein pramoxine is in the amount of about 0.1% to about 10% weight of the composition (wt %). In other embodiments, pramoxine is in the amount of about 0.1 to about 5 wt %. In yet another embodiment, pramoxine is in the amount of about 0.1 to about 1 wt %. In other embodiments, pramoxine is in the amount of about 1 wt %. In other embodiments, pramoxine is in the amount of about 0.5 wt %. In still other embodiments, pramoxine is in the amount of about 0.1 wt %. In some embodiments, the pramoxine is pramoxine hydrochloride.

Diphenhydramine (CAS RN: 58-73-1) is an antihistamine. In some embodiments, the disclosure provides a composition, wherein diphenhydramine is in the amount of about 0.1% to about 10% weight of the composition (wt %). In other embodiments, diphenhydramine is in the amount of about 0.1 to about 5 wt %. In yet another embodiment, diphenhydramine is in the amount of about 0.1 to about 1 wt %. In other embodiments, diphenhydramine is in the amount of about 1 wt %. In other embodiments, diphenhydramine is in the amount of about 0.5 wt %. In still other embodiments, diphenhydramine is in the amount of about 0.1 wt %. In some embodiments, the diphenhydramine is diphenhydramine hydrochloride.

In some embodiments, the disclosure provides a composition wherein pramoxine is in the amount of about 0.1 to about 10 wt % and diphenhydramine is in the amount of about 0.1 to about 10 wt %. In some embodiments, the disclosure provides a composition wherein pramoxine is in the amount of about 1 wt % and diphenhydramine is in the amount of about 1 wt %.

Hydrocortisone (CAS RN: 50-23-7) is a steroid (corticosteroid) medicine. In some embodiments, the disclosure provides a composition, wherein hydrocortisone is in the amount of about 0.1 to about 5 wt %. In other embodiments, hydrocortisone is in the amount of about 0.5 to about 5 wt %. In other embodiments, hydrocortisone in the amount of about 0.1 to about 1 wt %. In another embodiment, hydrocortisone is in the amount of about 0.5 to about 1 wt %. In other embodiments, hydrocortisone is in the amount of about 0.5 wt %. In other embodiments, hydrocortisone is in the amount of about 0.1 wt %.

In some embodiments, the disclosure provides a composition wherein pramoxine is in the amount of about 0.1 to about 10 wt %, diphenhydramine is in the amount of about 0.1% to about 10 wt %, and hydrocortisone in the amount of about 0.1 to about 5 wt %. In some embodiments, the disclosure provides a composition wherein pramoxine is in the amount of about 1 wt %, diphenhydramine is in the amount of about 1 wt %, and hydrocortisone is in the amount of about 0.5 wt %.

In some embodiments, the composition further comprises one or more of allantoin, licorice root extract, and a polysaccharide. In some embodiments, the polysaccharide is a natural branched polysaccharide, optionally a rhamnose-rich polysaccharide, obtained by bacterial fermentation. In some embodiments, the polysaccharide is TEFLOSER (available from Solabia Group, Pantin, France).

In some embodiments, the disclosure provides a composition, wherein allantoin is in the amount between about 0.01 to about 5 wt %, the licorice root extract is in the amount between about 0.05 to about 5 wt %, and the polysaccharide is in the amount between about 0.01 to about 5 wt %.

In some embodiments, the disclosure provides a composition wherein pramoxine is in the amount of about 1 wt %, diphenhydramine is in the amount of about 1 wt %, and hydrocortisone is in the amount of about 0.5 wt %, and the composition further comprises propylene glycol, water, SD alcohol 40-B, glycerin, polysorbate 80, benzyl alcohol, PEG-75 lanolin, allantoin, hydrolyzed oats, Glycyrrhiza glabra (licorice) root extract, propanediol, rhamnose, glucose, glucuronic acid, and sodium hydroxide.

In some embodiments, the disclosure provides a composition wherein pramoxine is in the amount of about 0.5 wt %, diphenhydramine is in the amount of about 0.5 wt %, and hydrocortisone is in the amount of about 0.1 wt %, and the composition further comprises propylene glycol, water, SD alcohol 40-B, glycerin, polysorbate 80, benzyl alcohol, PEG-75 lanolin, allantoin, hydrolyzed oats, Glycyrrhiza glabra (licorice) root extract, propanediol, rhamnose, glucose, glucuronic acid, and sodium hydroxide.

Allantoin (CAS RN: 97-59-6) promotes and accelerates cell proliferation and provides keratolytic and soothing action and has a beneficial influence on skin moisture. In some embodiments, the disclosure provides a composition, wherein allantoin is in the amount of about 0.01 to about 5 wt %. In other embodiments, allantoin is in the amount of about 0.1 to about 5 wt %. In another embodiment, allantoin is in the amount of about 0.1 to about 1 wt %. In other embodiments, allantoin is in the amount of about 0.15 wt %.

Glycyrrhiza glabra (licorice) root extract is the polysaccharide fraction of licorice and has immunomodulatory activity by strengthening phagocytosis in the endothelial reticular system and stimulating interferon production. In some embodiments, the disclosure provides a composition, wherein the licorice root extract is in the amount of about 0.05 to about 5 wt %. In some embodiments, the disclosure provides a composition, wherein the licorice root extract is in the amount of about 0.05 to about 1 wt %. In some embodiments, the disclosure provides a composition, wherein the licorice root extract is in the amount of about 0.1 to about 1 wt %. In other embodiments, the licorice root extract is in the amount of about 0.25 to about 1 wt %. In other embodiments, the licorice root extract is in the amount of about 0.25 wt %.

Polysaccharides such as rhamnose, glucose, glucuronic acid, alpha-glucan oligosaccharide protect microbiota equilibrium, inhibit biofilm formation and prevents adhesion of undesirable and/or pathogenic bacteria on skin's surface. In some embodiments, the polysaccharide is in the amount of about 0.01 to about 5 wt %. In other embodiments, the polysaccharide is in the amount of about 0.01 to about 1 wt %. In other embodiments, the polysaccharide is in the amount of about 0.05 to about 1 wt %. In other embodiments, the polysaccharide is in the amount of about 0.05 wt %.

Hydrolyzed oats (CAS RN: 227025 Aug. 3) is a soluble form of oats, derived from whole oats by hydrolysis, and is a humectant having a beneficial influence on skin moisture.

Propanediol (1,3-propanediol) (CAS RN: 504-63-2) is a solvent and emollient derived from corn sugar.

PEG-75 lanolin (CAS RN: 61790-81-6) is a polyoxyethylene condensate, with pharmaceutical grade lanolin content being about 17%.

In some embodiments, the disclosure provides a composition wherein pramoxine is in the amount of about 1 wt %, diphenhydramine is in the amount of about 1 wt %, allantoin is in the amount of about 0.15 wt %, licorice root extract is in the amount of about 0.25 wt %, and the polysaccharide is in the of amount of about 0.05 wt %.

In some embodiments, the disclosure provides a composition wherein pramoxine is in the amount of about 1 wt %, diphenhydramine is in the amount of about 1 wt %, hydrocortisone is in the amount of about 0.5 wt %, allantoin is in the amount of about 0.15 wt %, licorice root extract is in the amount of about 0.25 wt %, and the polysaccharide is in the amount of about 0.05 wt %.

In some embodiments, the disclosure provides a composition wherein pramoxine is in the amount of about 1 wt %, diphenhydramine is in the amount of about 1 wt %, hydrocortisone is in the amount of about 0.5 wt %, allantoin is in the amount between about 0.01 to about 5 wt %, and the licorice root extract is in the amount between about 0.05 to about 5 wt %.

In other embodiments, the composition may comprise additional ingredients as appropriate that would be well known to one of ordinary skill in the art such as penetration enhancers, humectants, lubricants, color, fragrance, preservatives, antioxidants, chelators, neutralizers, astringents, binders, catalysts, stabilizers, emollients, emulsifiers, surfactants, essential oils, plant/botanical extracts, conditioners, film formers, gelling agents, foaming agents, exfoliants, vitamins, minerals, pH adjusters, proteins, peptides, tactile enhancers, saccharides, solvents or any combination thereof.

In some embodiments, the composition disclosed herein may be formulated as a wipe, spray, cream, lotion, gel, fluid/liquid, soap, or any other form known in the art. In some embodiments, the composition is formulated as a wipe.

In embodiments wherein the composition disclosed herein is formulated as a wipe, a wide variety of materials can be used as the wipe. Examples of suitable wipe substrates include, but are not limited to, woven fabrics, non-woven fabrics such as needle-punched fabrics, hydro-entangled fabrics, or other entangled fabrics. The wipe is preferably formed to retain the composition (such as by absorbing the composition among, along, and/or between fibers of the wipe) for a period of time at least as long as from when the product is manufactured to a time when the product is used by a consumer (i.e., a shelf storage period).

In some embodiments, the disclosure provides methods of treating or preventing a skin condition, the methods comprising topically administering to a subject in need of such treatment an effective amount of one or more of compositions of the disclosure. In some embodiments, the skin condition is a result of an allergy. Allergic conditions of the skin include but are not limited to Atopic Dermatitis, Flea Allergy Dermatitis, Contact Allergy Dermatitis, Food Allergies, Food Sensitivity, and the like. In other embodiments, the skin condition is a bacterial, fungal or parasitic infection of the skin. Any disclosed composition, or a combination of compositions may be used in these methods.

The disclosure provides methods as described above, wherein the subject is a mammalian subject. In one embodiment, the subject is a pet. In one embodiment, the subject is a dog or a cat. In another embodiment, the subject is a dog. In other embodiment, the subject is a cat.

In some embodiments, in the methods as described above, the composition is administered over the course of a period of time. In some embodiments, the composition may be applied on a daily basis, either once a day or several times per day, over the course of days, weeks, months or years. In some embodiments, in the methods as described above, the administration is episodically, for example on an as needed basis when the subject is experiencing a skin condition for a short-term period of several months.

The compositions described herein will generally be used in an amount effective to achieve the intended result, for example in an amount effective to treat or prevent a skin condition. By therapeutic benefit it is meant eradication or amelioration of the skin condition and/or eradication or amelioration of one or more of the symptoms associated with a skin condition such that the subject shows an improvement in feeling or condition, notwithstanding that the subject may still be afflicted with the condition. Therapeutic benefit also generally includes halting or slowing the progression of the skin condition, regardless of whether improvement is realized.

The compositions described herein may be topically applied or delivered, in an easy to use manner (including easy to apply), and that are both effective in treating the intended region of skin and gentle enough so as not to irritate sensitive skin.

### Examples

The compositions and methods of the disclosure are further illustrated by the following examples, which are not to be construed as limiting the disclosure in scope or spirit to the specific procedures and compositions described in them.

A composition is formulated into a wipe by mixing the ingredients at room temperature to obtain a solution, followed by soaking an absorbent wipe substrate in the solution until the solution is absorbed onto the wipe. Representative compositions that are present per wipe are listed in Table 1, Table 2, Table 3, and Table 4.

TABLE 1
3-in-1 Medicated Wipe

| Actives | Amt (mg/wipe) | wt % |
|---|---|---|
| Pramoxine HCl | | 1.00% |
| Diphenhydramine | | 1.00% |
| Allantoin | 0.000061 | 0.15% |
| Glycyrrhiza Glabra (Licorice) Root Extract | 0.000305 | 0.25% |
| Polysaccharides (Teflose) | 0.000183 | 0.05% |

TABLE 2
3-in-1 Medicated Wipe

| Actives | Amt (mg/wipe) | wt % |
|---|---|---|
| Pramoxine HCl | | 1.00% |
| Diphenhydramine | | 1.00% |
| Hydrocortisone | | 0.50% |
| Allantoin | 0.000061 | 0.15% |
| Glycyrrhiza Glabra (Licorice) Root Extract | 0.000305 | 0.25% |
| Polysaccharides (Teflose) | 0.000183 | 0.05% |

TABLE 3
3-in-1 Medicated Wipe

| Actives | Amt (mg/wipe) | wt % |
|---|---|---|
| Pramoxine HCl | | 1.00% |
| Diphenhydramine | | 1.00% |
| Hydrocortisone | | 0.50% |

TABLE 4
3-in-1 Medicated Wipe

| Actives | Amt (mg/wipe) | wt % |
|---|---|---|
| Pramoxine HCl | | 0.50% |
| Diphenhydramine | | 0.50% |
| Hydrocortisone | | 0.10% |

In the composition of Tables 3 and 4, additional ingredients comprising propylene glycol, water, SD alcohol 40-B, glycerin, polysorbate 80, benzyl alcohol, PEG-75 lanolin, allantoin, hydrolyzed oats, Glycyrrhiza glabra (licorice) root extract, propanediol, rhamnose, glucose, glucuronic acid, and sodium hydroxide may be present in the medicated wipe. In some examples, allantoin may be present at 0.15% by weight. In some examples, Glycyrrhiza glabra (licorice) root extract may be present at 0.25% by weight.

It is understood that the examples and embodiments described herein are for illustrative purposes only. Unless clearly excluded by the context, all embodiments disclosed for one aspect of the invention can be combined with embodiments disclosed for other aspects of the invention, in any suitable combination. It will be apparent to those skilled in the art that various modifications and variations can be made to the present invention without departing from the scope of the invention. Thus, it is intended that the present invention cover the modifications and variations of this invention provided they come within the scope of the appended claims and their equivalents.

1. A composition comprising pramoxine in the amount of about 0.1% to about 10% by weight of the total composition and diphenhydramine in the amount of about 0.1% to about 10% by weight of the total composition.

2. The composition of claim 1 further comprising hydrocortisone in an amount between about 0.1% to about 5% by weight of the total composition.

3. The composition of claim 2, wherein pramoxine is in the amount of about 1% by weight of the total composition, diphenhydramine is in the amount of about 1% by weight of the total composition, and hydrocortisone is in the amount of about 0.5% by weight of the total composition.

4. The composition of claim 1, wherein pramoxine is pramoxine hydrochloride.

5. The composition of claim 1, wherein diphenhydramine is diphenhydramine hydrochloride.

6. The composition of claim 1further comprising one or more of allantoin, licorice root extract, and polysaccharide.

7. The composition of claim 6, wherein allantoin is an amount between about 0.0 1% to about 5% by weight of the total composition, the licorice root extract is an amount between about 0.05% to about 5% by weight of the total composition, and the polysaccharide is an amount between about 0.01% to about 5% by weight of the total composition.

8. The composition of claim 6, wherein pramoxine is in the amount of about 1% by weight of the total composition, diphenhydramine in is in the amount of about 1% by weight of the total composition, hydrocortisone in is the amount of about 0.5% by weight of the total composition, allantoin is in the amount of about 0.15% by weight of the total composition, the licorice root extract is in the amount of about 0.25% by weight of the total composition, and the polysaccharide is in the amount of about 0.05% by weight of the total composition.

9. The composition of claim 1, further comprising one or more of propylene glycol, SD alcohol 40-B, glycerin, polysorbate 80, benzyl alcohol, PEG-75 lanolin, hydrolyzed oats, propanediol, glucose, glucuronic acid, and sodium hydroxide.

10. The composition of claim 1, wherein the composition is formulated as an absorbent article.

11. The composition of claim 1, wherein the composition is formulated as a wipe.

12. A method of treatment or prevention of a skin condition, the method comprising administering to a subject in need of such treatment an effective amount of one or more compositions of claim 1.

13. The method of claim 12, wherein the subject is a dog or a cat.

14. The method of claim 12, wherein the skin condition is an infection.

15. The method of claim 12, wherein the skin condition is a result of an allergy.
